(19) United States
(12) Patent Application Publication
(10) Pub. No.: US 2021/0244284 A1
(43) Pub. Date: Aug. 12, 2021

(54) EXAMINATION HEAD AND PERIODONTAL DISEASE EXAMINATION APPARATUS

(71) Applicant: TANITA CORPORATION, Tokyo (JP)

(72) Inventor: Mikio SHINDO, Tokyo (JP)

(21) Appl. No.: 17/219,364

(22) Filed: Mar. 31, 2021

Related U.S. Application Data
(63) Continuation of application No. PCT/JP2019/038858, filed on Oct. 2, 2019.

(30) Foreign Application Priority Data
Oct. 4, 2018 (JP) 2018-189239

(51) Int. Cl. A61B 5/00

(52) U.S. Cl. CPC A61B 5/0088; A61B 5/0079; A61B 2562/0233; A61B 5/0066; A61B 5/742

(57) ABSTRACT

In an examination head used in an apparatus for examining periodontal disease, measuring light beams, which are parallel light beams, are emitted from a light-emitting surface of a light-emitting member. A reflecting member on which a reflecting surface is formed is provided at a position opposing the light-emitting surface of the light-emitting member. With a tooth and gum sandwiched by the light-emitting surface and reflecting surface, the measuring light beams to are emitted. Interference signals are generated utilizing light beams which are a reflection of the measuring light beams to from the reflecting surface in addition to light beams which are a reflection of the measuring light beams to from the tooth and gum. Interference signals having an excellent S/N ratio are obtained.

### TECHNICAL FIELD

This invention relates to an examination head and a periodontal disease examination apparatus.

### BACKGROUND ART

Measurement of the depth of a periodontal pocket is carried out as one example of an examination for periodontal disease. In general, the depth of a periodontal pocket is measured visually as by a dentist inserting a rod-like measuring instrument referred to as a “pocket probe” into the periodontal pocket. However, there are occasions where the result of measurement is not necessarily accurate owing to the level of skill of the dentist or the like, the angle of insertion of the pocket probe and visual error, etc. Further, there is concern that, owing to bleeding from the gums at the time of examination, affected parts free of periodontal disease will become infected with periodontal disease. For these reasons, consideration has been given to an OCT (Optical Coherence Tomography) apparatus for irradiating a specimen with light to thereby acquire information about the oral cavity from tomographic information (Patent Document 1).

### PRIOR-ART DOCUMENTS

### Patent Documents

Patent Document 1: Japanese Patent Application Laid-Open No. 2013-257166

However, since measuring light emitted from an OCT apparatus repeatedly undergoes transmission, reflection and interference up to the limit of transmission, the transmitted light attenuates as it advances through the interior of the specimen. As a consequence, the thicker the specimen, the worse the S/N ratio of the interference signal obtained. Hence there are instances where accurate information about the specimen is not obtained.

### DISCLOSURE OF THE INVENTION

An object of the present invention is to prevent a decline in the S/N ratio of an interference signal caused by an increase in the thickness of a specimen.

According to a first aspect of the present invention, an examination head used in an apparatus for examining periodontal disease by utilizing measuring light and reference light split off from low-interference light is characterized by having a holder formed to include a light-emitting surface for emitting measuring light which is parallel light, and a reflecting surface parallel to (or substantially parallel to) the light-emitting surface for reflecting the measuring light emitted from the light-emitting surface.

The light-emitting surface and the reflecting surface may be formed on a light-emitting member and on a reflecting member, respectively, the holder holding the light-emitting member and the reflecting member.

By way of example, the holder supports the light-emitting member and the reflecting member such that the light-emitting surface and reflecting surface are capable of approaching each other and of separating from each other while maintaining a parallel state.

The examination head further has a rod-shaped gripping member one end portion of which is secured to the holder. In this case, by way of example, the gripping member is freely foldable through a predetermined angle, a straight line in a direction identical with that of the optic axis of the measuring light serving as an axis of rotation.

The examination head may further have a first transfer member for transferring a force applied to at least one of the light-emitting member and reflecting member in such a manner that the light-emitting surface and reflecting surface approach each other, and may further have a second transfer member for transferring a force applied to at least one of the light-emitting member and reflecting member in such a manner that the light-emitting surface and reflecting surface separate from each other.

The examination head may further have a tension member for pulling the light-emitting member and the reflecting member in such a manner that the light-emitting surface and reflecting surface approach each other, and may further have a compression member for applying a force that separates the light-emitting member and the reflecting member in such a manner that the light-emitting surface and reflecting surface separate from each other.

The holder is, for example, a mouthpiece placed in close contact with a surface portion of teeth at a boundary with gums and with a portion of the gums, and comprises a flexible material. In this case, the mouthpiece preferably is formed to have a cavity into which the teeth and a portion of the gums penetrate owing to placement of the mouthpiece, the light-emitting surface is formed on one surface of two surfaces that oppose each other sandwiching between them the teeth in the cavity, and the reflecting surface is formed on the other surface of the two surfaces.

The reflecting surface is, for example, a front-surface mirror or a back-surface mirror.

A periodontal disease examination apparatus according to a second aspect of the present invention comprises: the above-described examination head; an optical divider for splitting low-interference light into measuring light and reference light; a parallelizing element for rendering as parallel light the measuring light split off by the optical divider; an optical waveguide for guiding the measuring light, which has been rendered as parallel light by the parallelizing element, to the examination head and causing the measuring light to be emitted from the light-emitting surface; a photodetector for outputting an interference signal obtained by detecting reflected light, which is light reflected from a gum or tooth owing to irradiation of the gum or tooth with the measuring light emitted from light-emitting surface of the examination head, reflected light which is a result of the emitted light emitted from light-emitting surface of the examination head being reflected from the reflecting surface of the examination head, and reflected light which is a result of the reference light split off by the optical divider being reflected by a reference surface; and periodontal pocket data generating means for generating data regarding depth of a periodontal pocket based on the interference signal output from the photodetector.

In accordance with the first aspect of the present invention, since the reflecting surface is formed parallel to the light-emitting surface, measuring light reflected from the reflecting surface is obtained. If an interference signal is generated using not only the measuring light emitted from the light-emitting surface but also the measuring light that is a reflection from the reflecting surface, it is possible to mitigate a decline in the S/N ratio of the interference signal caused by an increase in the thickness of the specimen. In accordance with the second aspect of the present invention, due to the fact that an interference signal is generated using not only the measuring light emitted from the light-emitting surface but also the measuring light that is a reflection from the reflecting surface, it is possible to mitigate a decline in the S/N ratio of the interference signal caused by an increase in the thickness of the specimen, and comparatively accurate date is obtained regarding a periodontal pocket is obtained.

### BRIEF DESCRIPTION OF THE DRAWINGS

FIG. 1 is a block diagram illustrating the electrical configuration of a periodontal disease examination apparatus;

FIG. 2 illustrates the construction of a deflecting unit;

FIG. 3 illustrates the construction of the deflecting unit;

FIG. 4 illustrates the manner in which a gum and a tooth are irradiated with measuring light;

FIG. 5A to FIG. 5E are examples of interference signals;

FIG. 6 is an example of optical tomographic images;

FIG. 7 is a perspective view of an examination head;

FIG. 8 is a sectional view taken along line VIII-VIII of FIG. 7;

FIG. 9 is a sectional view taken along line IX-IX of FIG. 7;

FIG. 10 is a perspective view of an examination head;

FIG. 11 is a sectional view taken along line XI-XI of FIG. 10;

FIG. 12 illustrates that manner in which a gripping portion of an examination head is folded;

FIG. 13 is a sectional view of an examination head;

FIG. 14 is a sectional view of an examination head;

FIG. 15 is a perspective view of a light-emitting member;

FIG. 16 is a perspective view of a mouthpiece as well as teeth enveloped by gums;

FIG. 17 illustrates the manner in which the mouthpiece is placed on teeth;

FIG. 18 is a plan view of the mouthpiece;

FIG. 19 is a sectional view taken along line XIX-XIX of FIG. 18;

FIG. 20 is a plan view of the mouthpiece placed on teeth;

FIG. 21 is a sectional view taken along line XXI-XXI of FIG. 18; and

FIG. 22 is a sectional view taken along line XXII-XXII of FIG. 20.

### BEST MODE FOR CARRYING OUT THE INVENTION

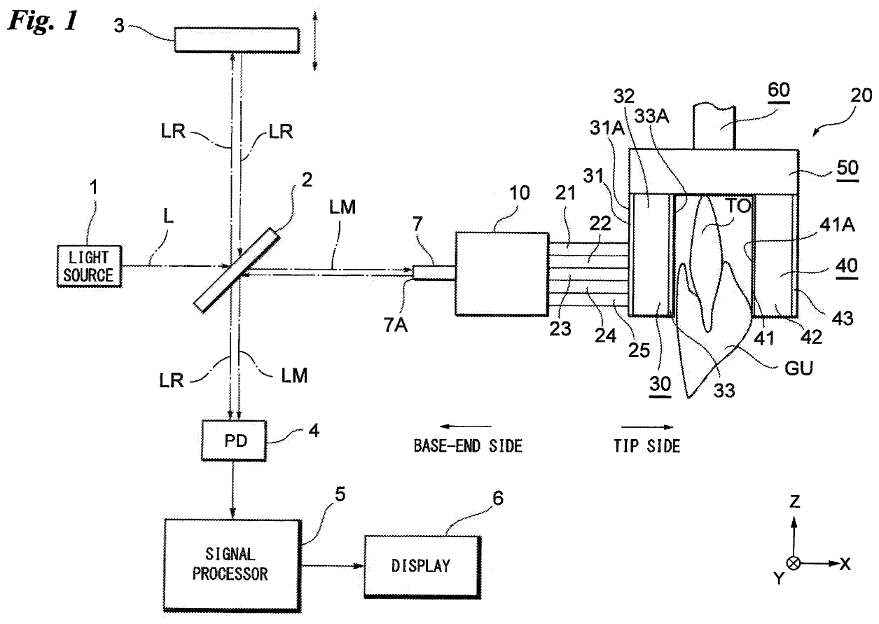

FIG. 1, which illustrates an embodiment of the present invention, is a block diagram showing the construction of a periodontal disease examination apparatus.

Low-interference light (low-coherence light) L is emitted from a light source 1 such as an SLD (Super Luminescent Diode). The low-interference light L is split into measuring light LM and reference light LR by a beam splitter 2 (one example of an optical divider). It will suffice if low-interference light L is emitted from the light source 1, and use may be made of another light source such as a gas laser, semiconductor laser or laser diode.

The measuring light LM split off by the beam splitter 2 impinges on a first optical fiber 7 from a light-incident end face 7A of the first optical fiber 7. (The light-incident end face impinged by the measuring light LM is the end face on the base-end side in FIG. 1). A light-emitting end face for emitting the measuring light LM of the first optical fiber 7 (the light-emitting end face that emits the measuring light LM is the end face on the tip side in FIG. 1) is connected to a deflecting unit 10. Connected to the deflecting unit 10 are five (five is the number adopted for the sake of convenience but the number may be more or less than five) second optical fibers 21 to 25. The measuring light LM emitted from the measuring-light-LM light-emitting end face of the first optical fiber 7 is deflected, and is rendered parallel, by the deflecting unit 10 so as to impinge successively on each of measuring-light-LM-incident end faces of the five optical fibers 21 to 25 (example of waveguides).

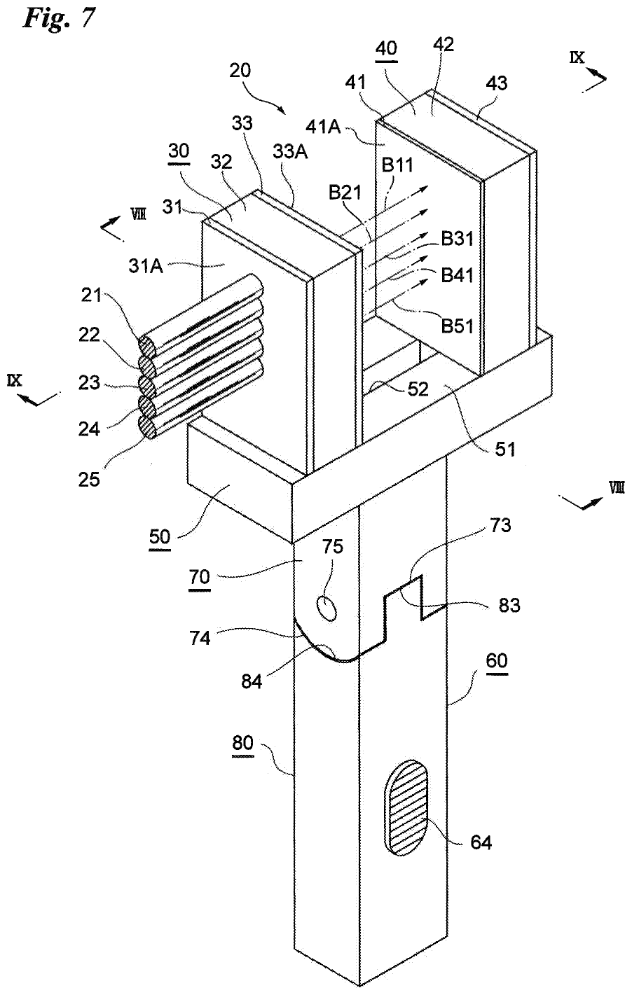

An examination head 20 (see FIGS. 7 to 12, etc. for the details) of the periodontal disease examination apparatus includes a holder 50. A light-emitting member 30 for emitting the measuring light LM is formed on the holder 50. As will be described in detail later (see FIG. 7, etc.), the light-emitting member 30 is composed of two plate-shaped members 31 and 33 and a deforming member 32. Held on the holder 50 are the light-emitting member 30 and a reflecting member 40 formed to have a reflecting surface 41A which reflects the measuring light LM emitted from a light-emitting surface 33A of the light-emitting member 30. As will be described in detail later (see FIG. 7, etc.), the reflecting member 40 is composed of a sheet-like mirror 41, a deforming member 42 and a plate-shaped member 43. The light-emitting member 30 and the reflecting member 40 are held on the holder 50 in such a manner that the light-emitting surface 33A of the light-emitting member 30 and the reflecting surface 41A of the reflecting member 40 are made parallel. A gripping member 60 by which the user grips the examination head 20 is secured to the holder 50. The portions of the second optical fibers 21 to 25 on the side of their measuring-light-LM light-emitting end faces penetrate into the light-emitting member 30 from a light-incident surface 31A of the light-emitting member 30 and are secured inside the light-emitting member 30.

The measuring light LM that has impinged on the second optical fibers 21 to 25 propagates through the second optical fibers 21 to 25 and is emitted from the light-emitting surface 33A of the light-emitting member 30. The measuring light LM irradiates a gum GU and a tooth TO which are to undergo measurement.

Some of the measuring light LM that has irradiated the gum GU and tooth TO to undergo measurement is reflected from the gum GU and tooth TO. The measuring light LM reflected from the gum GU and tooth TO passes through the second optical fibers 21 to 25 and is guided to the first optical fiber 7 by the deflecting unit 10.

Further, some of the measuring light LM is transmitted through the gum GU and tooth TO and is reflected at the reflecting surface 41A of the reflecting member 40. The measuring light LM reflected from the reflecting surface 41A also is transmitted through the gum GU and tooth TO, passes through the second optical fibers 21 to 25 and is guided to the first optical fiber 7 by the deflecting unit 10.

The measuring light LM reflected from the gum GU and tooth TO and the measuring light LM reflected from the reflecting surface 41A returns from the first optical fiber 7, is reflected in the beam splitter 2 and impinges upon a photodiode 4 (one example of a photodetector).

Further, the reference light LR split off in the beam splitter 2 is reflected at a reference mirror 3 (reference surface) freely movable along the direction of propagation of the reference light LR and in the direction opposite thereto (along the positive and negative directions of the Z-axis in the embodiment shown in FIG. 1). The reflected reference light LR is transmitted through the beam splitter 2 and impinges upon the photodiode 4.

When, by moving the reference mirror 3, equality is established between a propagation distance, which is the sum total of propagation distance traveled until the measuring light LM irradiates the gum GU and tooth TO undergoing examination and propagation distance traveled until light reflected from the gum GU and tooth TO undergoing examination impinges upon the photodiode 4, and a propagation distance, which is the sum total of propagation distance traveled until the reference light LR irradiates the reference mirror 3 and light reflected from the reference mirror 3 impinges upon the photodiode 4, interference occurs between the measuring light LM and reference light LR and the photodiode 4 outputs an interference signal.

The interference signal output from the photodiode 4 is input to a signal processing circuit 5 (one example of a periodontal pocket data generating device), and signals representing optical tomographic images of the gum GU and tooth TO (data regarding the depth of a periodontal pocket) are generated. By inputting the generated signals representing the optical tomographic images to a display unit 6, the optical tomographic images of the gum GU and tooth TO are displayed on the display screen of the display unit 6. Processing for extracting the contours of the optical tomographic images is executed in the signal processing circuit 5, whereby the depth of a periodontal pocket between the gum GU and tooth TO is calculated. The calculated depth of the periodontal pocket also is displayed on the display screen of the display unit 6. Although optical tomographic images are generated and the depth of the periodontal pocket is calculated from the generated optical tomographic images, an arrangement may be adopted in which, rather than generate optical tomographic images, numerical data representing the depth of the periodontal pocket (such numerical data also is considered to be data regarding the depth of the periodontal pocket) is calculated in the signal processing circuit 5 and the depth of the periodontal pocket is displayed on the display screen of the display unit 6.

In this embodiment, with regard to the optical fibers 7, 21 to 25 and the like, the portion in the direction in which the measuring light LM is emitted is taken as the tip side, and the portion in the direction in which the measuring light LM is reflected back is taken as the base-end side.

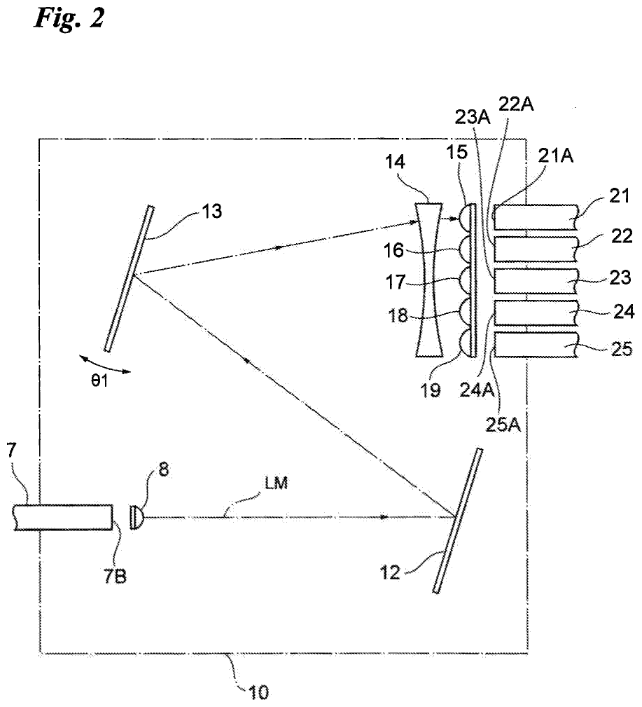

FIG. 2 illustrates the construction of the deflecting unit 10.

The first optical fiber 7 is connected to the deflecting unit 10, as mentioned above. A GRIN (gradient index) lens 11 is placed in front of the light-emitting end face 7B of the first optical fiber 7. (A GRIN lens is one example of a parallelizing element for outputting the incident light upon rendering it parallel. Another lens or optical element will also suffice as long as the incident light can be rendered parallel.) The measuring light LM rendered parallel by the GRIN lens 11 is reflected by a fixed mirror 12 (which does not rotate but which may be arranged to rotate) and is guided to a deflecting mirror 13. The deflecting mirror 13 is rotatable through a predetermined angle and causes the incident light to be reflected at a deflection angle conforming to the angle of rotation. A MEMS (Micro-Electro-Mechanical Systems) mirror, for example, is adopted as the deflecting mirror 13. The measuring light LM reflected at the deflecting mirror 13 is rendered parallel by an f-θ lens 14 (one example of a parallelizing element for outputting the incident light upon rendering it parallel; may just as well be another parallelizing element), passes through any of condensing lenses 15 to 19 and impinges on any of the second optical fibers 21 to 25 from the light-incident end faces 21A to 25A of the second optical fibers 21 to 25. It should be noted that the meaning of the term “parallelizing” is not limited to making light perfectly parallel but is a concept that also includes making light approximately parallel. Further, in this embodiment, it is preferred that the parallelizing element render light slightly condensed rather than perfectly parallel. That is, it is preferred that the effects of attenuation of light and of diffusion when light is transmitted through a substance be reduced, and that the focal point of the light not be situated in close proximity to the parallelizing element.

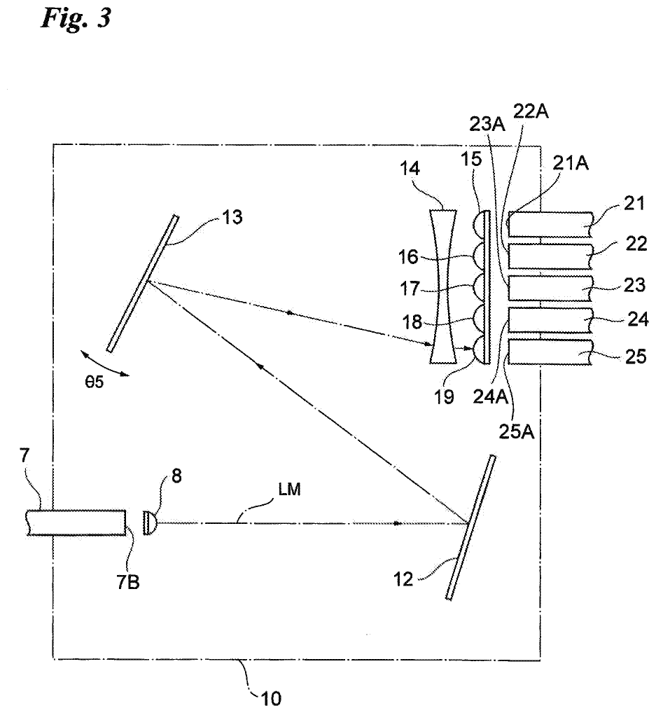

By controlling the angle of rotation of the deflecting mirror 13 using a control unit (not shown), the measuring light LM can be made to impinge on any of the second optical fibers 21 to 25. For example, by rotating the deflecting mirror 13 through an angle θ₁ from a predetermined angle, the measuring light LM will impinge on the second optical fiber 21 through the condensing lens 15, as illustrated in FIG. 2. Similarly, when the deflecting mirror 13 is rotated through an angle θ₂, θ₃ or θ₄ from a predetermined angle, the measuring light LM will impinge on the second optical fiber 22, 23 or 24 through the condensing lens 16, 17 or 18. When the deflecting mirror 13 is rotated through an angle θ₅ from a predetermined angle, the measuring light LM will impinge on the second optical fiber 25 through the condensing lens 19, as shown in FIG. 3.

The measuring light LM emitted from the measuring-light-LM light-emitting end faces of the second optical fibers 21 to 25 (the measuring light LM that has been rendered parallel is thus guided to the examination head 20 in the second optical fibers 21 to 25) is reflected at the gum GU and tooth TO as well as at the reflecting surface 41A and again impinges on the second optical fibers 21 to 25 from the light-emitting end faces thereof that emitted the light. The measuring light LM that has again impinged on the second optical fibers 21 to 25 after being reflected at the gum GU and tooth TO is again incident upon the first optical fiber 7 via a path that is the reverse of the path on which the light is emitted from the first optical fiber 7 to the second optical fibers 21 to 25.

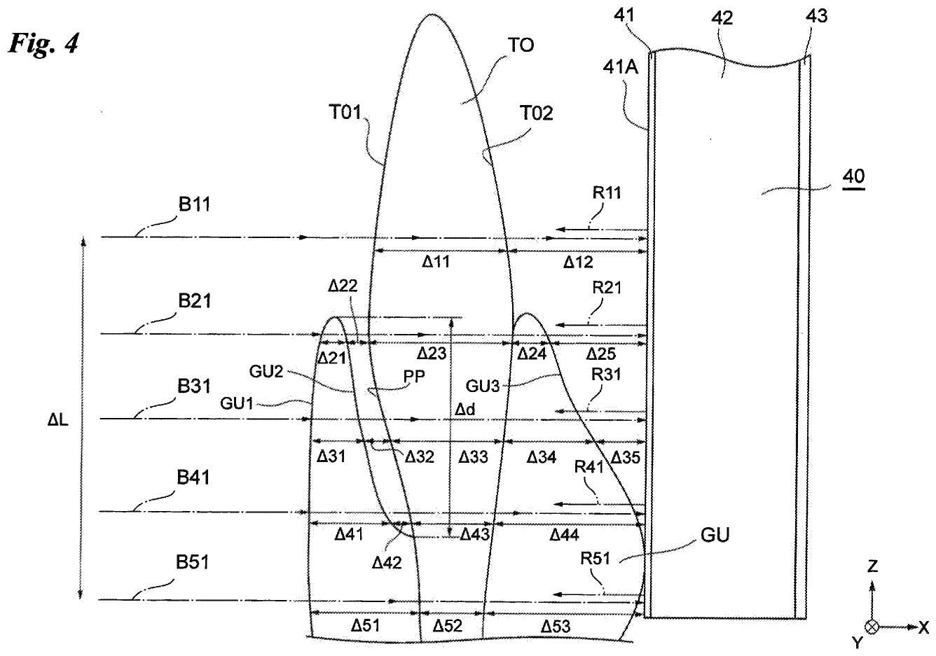

FIG. 4 illustrates the manner in which the gum GU and the tooth TO undergoing examination are irradiated with measuring light beams B11, B21, B31, B41 and B51. In FIG. 4 the illustration of the light-emitting member 30 is omitted. Among the beams of measuring light LM, the beams of measuring light LM emitted from the second optical fibers 21, 22, 23, 24 and 25 are represented by measuring light beams B11, B21, B31, B41 and B51, respectively, and the measuring light beams B11, B21, B31, B41 and B51 reflected at the reflecting surface 41A are represented by R11, R12, R13, R14 and R15, respectively.

The measuring light beam B11 is the measuring light LM that propagates through the second optical fiber 21. Similarly, the measuring light beams B21, B31, B41 and B51 are beams of the measuring light LM that propagate through the second optical fibers 22, 23, 24 and 25, respectively.

FIG. 4 illustrates the gum GU and tooth TO as seen from the side. The left side in FIG. 4 corresponds to one of either the outside or the inside of the body, and the right side corresponds to the other one of either the outside or the inside of the body.

A periodontal pocket PP has formed between the gum GU and tooth TO. In the case of severe periodontal disease, the depth of the periodontal pocket PP is 6 mm or more. Therefore, if deflection width ΔL of the measuring light beams B11 to B51 (deflection width of the measuring light beams B11 to B51 along the depth direction of the periodontal pocket PP) is 6 mm or more, then whether the periodontal pocket PP exhibits severe periodontal disease can be determined. Accordingly, the number of second optical fibers 21 to 25 and the diameter of each of the second optical fibers 21 to 25 are decided in such a manner that the deflection width ΔL of the measuring light beams B11 to B51 will be 6 mm or more. Thus, enough deflection width to measure the depth of a periodontal pocket in a single scan is preferred.

A portion of the measuring light beam B11 is reflected at surface TO1 of the tooth TO and at inner surface TO2 on the back side of the tooth TO, and these reflected light beams are introduced to the second optical fiber 21. Similarly, a portion of the measuring light beam B21 is reflected at surface GU1 of the gum GU on the left side in FIG. 4, at inner surface GU2 on the back side of the gum GU on the left side, at the surface TO1 of the tooth TO, at inner surface TO2 on the back side of the tooth TO, and at inner surface GU3 on the back side of the gum GU on the right side, and these reflected light beams are introduced to the second optical fiber 22. Similarly, portions of respective measuring light beams B21 and B31 are reflected at the surface GU1 of the gum GU on the left side in FIG. 4, at the inner surface GU2 on the back side of the gum GU on the left side, at the surface TO1 of the tooth TO, at inner surface TO2 on the back side of the tooth TO, and at the inner surface GU3 on the back side of the gum GU on the right side, and these reflected light beams are introduced to the respective second optical fibers 22 and 23. A portion of the measuring light beam B51 is reflected at the surface GU1 of the gum GU on the left side in FIG. 4, at the inner surface GU2 on the back side of the gum GU on the left side (this surface is the same as the surface TO1 of the tooth TO since there is no periodontal pocket on the back side of the tooth TO), at the inner surface TO2 on the back side of the tooth TO and at the inner surface GU3 on the back side of the gum GU on the right side, and these reflected light beams are introduced to the second optical fiber 22.

In FIG. 4, a reflected light beam R11, which is a light beam obtained when the measuring light beam B11 that has passed through the tooth TO is reflected at the reflecting surface 41A, passes through the tooth TO and is introduced to the second optical fiber 21. A reflected light beam R12, which is a light beam obtained when the measuring light beam B21 that has passed through the gum GU and the tooth TO is reflected at the reflecting surface 41A, passes through the gum GU and the tooth TO and is introduced to the second optical fiber 22. Similarly, reflected light beams R13, R14 and R15, which are light beams obtained when the respective measuring light beams B31, B41 and B51 are reflected at the reflecting surface 41A, pass through the gum GU and the tooth TO and are introduced to the second optical fibers 23, 24 and 25, respectively.

In this embodiment, in addition to the reflected light beams obtained owing to reflection of the measuring light beams B11 to B51 at the tooth TO, etc., the reflected light beams R11 to R51, which are obtained when the respective measuring light beams are reflected at the reflecting surface 41A, are introduced to the respective second optical fibers 21 to 25. The reflected light beams that have propagated through the respective second optical fibers 21 to 25 are emitted from the left-side end face 7A of the first optical fiber 7 as the reflected measuring light LM, as set forth above.

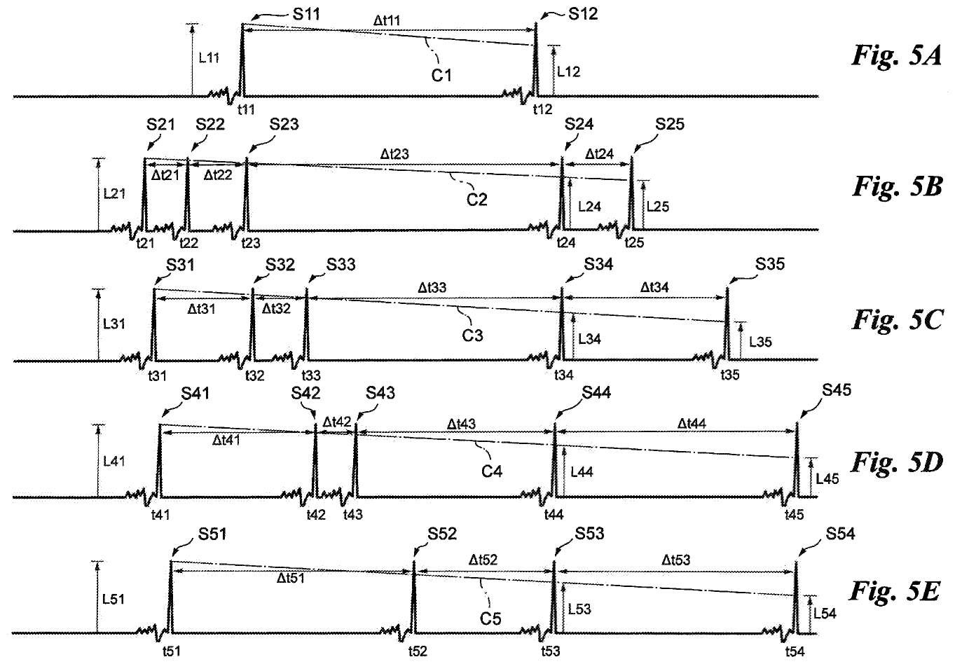

FIG. 5A to FIG. 5E are examples of interference signals.

FIGS. 5A, 5B, 5C, 5D and 5E are examples of interference signals obtained based on the measuring light beams B11, B21, B31, B41 and B51, respectively.

The measuring light beam B11 directly irradiates the portion of the tooth TO where the gum GU is not present (see FIG. 4), and the intensity of the light reflected from the surface of the tooth TO rises. Therefore, based on the light reflected from the surface TO1 on the left side of the tooth TO owing to the measuring light beam B11, an interference signal S11 of level L11 is generated at time til, as illustrated in FIG. 5A. Further, the measuring light beam B11 is reflected also at the inner surface TO2 on the back side of the tooth TO, and an interference signal S12 is generated at time t12, as shown in FIG. 5A, based on the measuring light beam B11. In actuality, since the measuring light beam B11 sustains attenuation and the like as it advances through the inside of the tooth TO that is the specimen, the amount of light reflected from the inner surface TO2 on the back side of the tooth TO is less than that of the light reflected from the surface TO1 on the left side of the tooth TO. If use were not made of the reflected light beam R11 from the reflecting surface 41A, therefore, the level of the interference signal S12 would be level L12, which is a level much lower in comparison with the level of the interference signal S11. (A chain line Cl is illustrated in order to facilitate understanding of the drop in level.) In this embodiment, however, the interference signals S11 and S12 are generated exploiting also the reflected light beam R11 from the reflecting surface 41A in addition to the reflection of the measuring light beam B11 at the inner surface TO2 on the back side of the tooth TO. In this embodiment, therefore, since the interference signal S12 is generated utilizing also the reflected light beam R11 from the reflecting surface 41A (the interference signal S11 also utilizes the reflected light beam R11 from the reflecting surface 41A), as illustrated in FIG. 5A, the level of the interference signal S12 is prevented from dropping by a large amount in comparison with the level L11 of the interference signal S11.

Since the measuring light beam B21 irradiates the upper end of the periodontal pocket PP (see FIG. 4), the intensity of the light reflected from the surface GU1 on the left side of the gum GU, from the inner surface GU2 on the back side of the gum GU on the left side, from the surface TO1 of the tooth TO, from the inner surface TO2 on the back side of the tooth TO and from the inner surface on the back side of the gum GU on the right side rises. As illustrated in FIG. 5B, therefore, interference signals 512, S22, S23, S24 and S25 are generated at times t21, t22, t23, t24 and t25, respectively, based on the light beams reflected from the surface GU1 on the left side of the gum GU, from the inner surface GU2 on the back side of the gum GU on the left side, from the surface TO1 of the tooth TO, from the inner surface TO2 on the back side of the tooth TO and from the inner surface on the back side of the gum GU on the right side, respectively. A time difference Δt21 from time t21 to time t22 indicates thickness Δ21 of the gum GU at the portion irradiated with the measuring light beam B21, a time difference Δt22 from time t22 to time t23 indicates distance Δ22 across the gap (the distance across the space between the tooth TO and gum GU) of the periodontal pocket PP at the portion irradiated with measuring light beam B21, a time difference Δt23 from time t22 to time t23 indicates thickness Δ23 of the tooth TO at the portion irradiated with the measuring light beam B21, and a time difference Δt24 from time t24 to time t25 indicates thickness Δ24 of the gum GU on the right side of the tooth TO at the portion irradiated with the measuring light beam B21.

If the interference signals S21, S22, S23, S24 and S25 were to be generated without utilizing the reflected light beam R21, then level L24 of the interference signal S24 and level L25 of the interference signal S25 in particular would drop greatly in comparison with interference signal S21, S22 or S23 (the drop in level is represented by chain line C2). In this embodiment, however, the interference signals S21, S22, S23, S24 and S25 are generated in the same manner as the interference signals S11 and S12 using the reflected light beam R21 of the measuring light beam B21. As illustrated in FIG. 5B, therefore, a large decline in the levels of the interference signals S24, S25, etc., is prevented.

Similarly, owing to the measuring light beam B31, there in a rise in the intensity of the light reflected from the surface GU1 on the left side of the gum GU, from the inner surface GU2 on the back side of the gum GU on the left side, from the surface TO1 of the tooth TO, from the inner surface TO2 on the back side of the tooth TO and from the inner surface on the back side of the gum GU on the right side. As illustrated in FIG. 5C, therefore, interference signals S31, S32, S33, S34 and S35 are generated at times t31, t32, t33, t34 and t35, respectively, based on the light beams reflected from the surface GU1 on the left side of the gum GU, from the inner surface GU2 on the back side of the gum GU on the left side, from the surface TO1 of the tooth TO, from the inner surface TO2 on the back side of the tooth TO and from the inner surface on the back side of the gum GU on the right side, respectively. A time difference Δt31 from time t31 to time t32 indicates thickness Δ31 of the gum GU at the portion irradiated with the measuring light beam B31, a time difference Δt32 from time t32 to time t33 indicates distance Δ32 across the gap (the distance across the space between the tooth TO and gum GU) of the periodontal pocket PP at the portion irradiated with measuring light beam B31, a time difference Δt33 from time t33 to time t34 indicates thickness Δ33 of the tooth TO at the portion irradiated with the measuring light beam B31, and a time difference Δt34 from time t34 to time t35 indicates thickness Δ34 of the gum GU on the right side of the tooth TO at the portion irradiated with the measuring light beam B31.

If the interference signals S31, S32, S33, S34 and S35 were to be generated without utilizing the reflected light beam R31, then level L34 of the interference signal S34 and level L35 of the interference signal S35 in particular would drop greatly in comparison with interference signal S31, S32 or S33 (the drop in level is represented by chain line C3). In this embodiment, however, the interference signals S31, S32, S33, S34 and S35 are generated in the same manner as the interference signals S11 and S12 using the reflected light beam R31 of the measuring light beam B31. As illustrated in FIG. 5C, therefore, a large decline in the levels of the interference signals S34, S35, etc., is prevented.

Similarly, owing to the measuring light beam B41, there in a rise in the intensity of the light reflected from the surface GU1 on the left side of the gum GU, from the inner surface GU2 on the back side of the gum GU on the left side, from the surface TO1 of the tooth TO, from the inner surface TO2 on the back side of the tooth TO and from the inner surface on the back side of the gum GU on the right side. As illustrated in FIG. 5D, therefore, interference signals S41, S42, S43, S44 and S45 are generated at times t41, t42, t43, t44 and t45, respectively, based on the light beams reflected from the surface GU1 on the left side of the gum GU, from the inner surface GU2 on the back side of the gum GU on the left side, from the surface TO1 of the tooth TO, from the inner surface TO2 on the back side of the tooth TO and from the inner surface on the back side of the gum GU on the right side, respectively. A time difference Δt41 from time t41 to time t42 indicates thickness Δ41 of the gum GU at the portion irradiated with the measuring light beam B41, a time difference Δt42 from time t42 to time t43 indicates distance Δ42 across the gap (the distance across the space between the tooth TO and gum GU) of the periodontal pocket PP at the portion irradiated with measuring light beam B41, a time difference Δt43 from time t43 to time t44 indicates thickness Δ43 of the tooth TO at the portion irradiated with the measuring light beam B41, and a time difference Δt44 from time t44 to time t45 indicates thickness Δ44 of the gum GU on the right side of the tooth TO at the portion irradiated with the measuring light beam B41.

If the interference signals S41, S42, S43, S44 and S45 were to be generated without utilizing the reflected light beam R41, then level L44 of the interference signal S44 and level L45 of the interference signal S45 in particular would drop greatly in comparison with interference signal S41, S42 or S43 (the drop in level is represented by chain line C4). In this embodiment, however, the interference signals S41, S42, S43, S44 and S45 are generated in the same manner as the interference signals S11 and S12 using the reflected light beam R41 of the measuring light beam B41. As illustrated in FIG. 5D, therefore, a large decline in the levels of the interference signals S44, S45, etc., is prevented.

Furthermore, owing to the measuring light beam B51, there in a rise in the intensity of the light reflected from the surface GU1 on the left side of the gum GU, from the inner surface GU2 on the back side of the gum GU on the left side (this surface is the same as the surface TO1 of the tooth TO because no periodontal pocket PP has formed), from the inner surface TO2 on the back side of the tooth TO and from the inner surface on the back side of the gum GU on the right side. As illustrated in FIG. 5E, therefore, interference signals S51, S52, S53 and S54 are generated at times t51, t52, t53 and t54, respectively, based on the light beams reflected from the surface GU1 on the left side of the gum GU, from the inner surface GU2 on the back side of the gum GU on the left side (from the surface TO1 of the tooth TO), from the surface TO1 of the tooth TO, from the inner surface TO2 on the back side of the tooth TO and from the inner surface on the back side of the gum GU on the right side, respectively. A time difference Δt51 from time t51 to time t52 indicates thickness Δ51 of the gum GU at the portion irradiated with the measuring light beam B51, a time difference Δt52 from time t52 to time t53 indicates thickness Δ53 of the tooth TO at the portion irradiated with the measuring light beam B51, and a time difference Δt53 from time t53 to time t54 indicates thickness Δ53 of the gum GU on the right side of the tooth TO at the portion irradiated with the measuring light beam B51.

If the interference signals S51, S52, S53 and S54 were to be generated without utilizing the reflected light beam R51, then level L53 of the interference signal S53 and level L54 of the interference signal S54 in particular would drop greatly in comparison with interference signal S51 or S52 (the drop in level is represented by chain line C5). In this embodiment, however, the interference signals S51, S52, S53 and S54 are generated in the same manner as the interference signals S11 and S12 using the reflected light beam R51 of the measuring light beam B51. As illustrated in FIG. 5E, therefore, a large decline in the levels of the interference signals S53, S54, etc., is prevented.

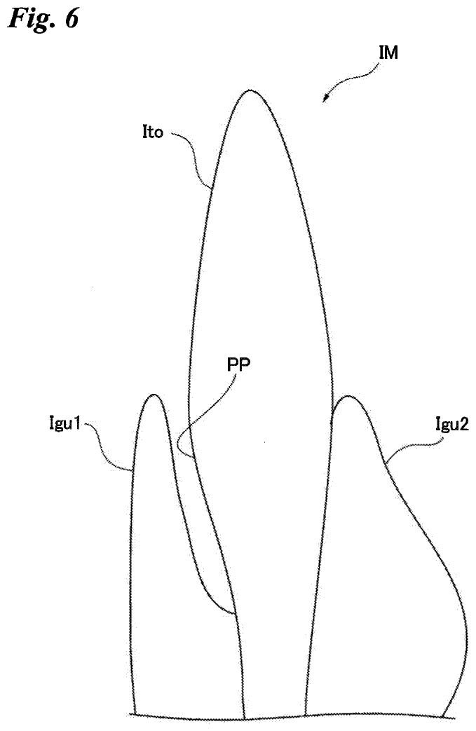

Optical tomographic images of the gum GU and tooth TO shown in FIG. 6 are generated by plotting the peak values of the interference signals of FIGS. 5A to 5E.

FIG. 6 is an example of an optical tomographic image IM.

The optical tomographic image IM includes an optical tomographic image Igu1 of the gum GU on the side irradiated with the measuring light beams B11 to B51, etc., an optical tomographic image Ito of the tooth TO, and an optical tomographic image Igu2 of the gum GU on the side of the gum GU opposite that irradiated with measuring light beams B11 to B51, etc. Such an optical tomographic image IM is displayed on the display screen of the display unit 6.

In a case where the above-mentioned interference signals S12 and the like are generated without utilizing the reflected light beams R11 to R51, often the optical tomographic image Igu2 of the gum GU on the side thereof opposite the surface impinged by the measuring light beams B11 to B51 will not be displayed clearly owing to attenuation of the measuring light beams B11 to B51. In this embodiment, however, interference signals S12, etc. having an excellent S/N ratio are generated utilizing the reflected light beams R11 to R51, thus resulting in the optical tomographic image IM. The optical tomographic image Igu2, therefore, is clearly displayed as well. When the Inventor actually produced an optical tomographic image using a dummy tooth and gum, a clear optical tomographic image was obtained by utilizing the reflected light beams R11 to R51.

By subjecting the optical tomographic image Igu1 of the gum GU and the optical tomographic image Ito of the tooth TO to contour extraction in the signal processing circuit 5, the depth Δd of the periodontal pocket PP is calculated in the signal processing circuit 5. In FIG. 6, a periodontal pocket has not formed between the optical tomographic image Igu2 and the optical tomographic image Ito of the tooth TO. However, in a case where a periodontal pocket has formed between the optical tomographic image Igu2 and the optical tomographic image Ito of the tooth TO, the depth of the periodontal pocket can be calculated.

In above-described embodiment, the depth Δd of the periodontal pocket PP is calculated by generating the optical tomographic image IM of the gum GU and tooth TO and extracting the contours of the optical tomographic image Igu1 and Ito in the generated optical tomographic image IM. However, the depth Δd of the periodontal pocket PP may be calculated by computation without generating the optical tomographic images Igu1 and Ito (although the optical tomographic images Igu1 and Ito may just as well be generated).

In the embodiment set forth above, it is assumed that the deflection width from the measuring light beam B11 to B51 is enough to enable measurement of the depth Δd of the periodontal pocket in a single scan even in case of severe periodontal disease. However, in instances where there is not enough deflection width to enable measurement of the depth Δd of the periodontal pocket in a single scan, an arrangement may be adopted in which, by performing measurement multiple times using the examination head 20 at positions that differ in height (at least at two locations), data regarding the depth Δd of the periodontal pocket will be generated in the signal processing circuit (a periodontal pocket data generating device) 5 based on interference signals output from the photodiode 4.

For example, assume that the light-emitting member 30 of the examination head 20 can emit measuring light having a deflection width corresponding to the range from measuring light beams B11 to B31 (equal to the range from B31 to B51), which is illustrated in FIG. 4, by a single scan (measurement). First, assume that a first scan (measurement) by the examination head 20 is carried out, at positions at which measuring light is capable of being emitted, over a range corresponding to the measuring light beams B11 to B31 illustrated in FIG. 4. In this case, the optical tomographic images Igu1, Igu2 and Ito of the upper half of gum GU and tooth TO shown in FIG. 4 are obtained from interference signals that are obtained based on measuring light emitted over the range from measuring light beams B11 to B31, shown in FIG. 4, in the first scan. Next, the examination head 20 is moved downward. Assume that, owing to a second scan performed at the position of the examination head after such movement, measuring light from the examination head 20 is emitted over the range corresponding to the measuring light beams B31 to B51 shown in FIG. 4. In this case, optical tomographic images Igu1, Igu2 and Ito of the lower half of gum GU and tooth TO shown in FIG. 4 are obtained from interference signals obtained based on measuring light emitted over the range from measuring light beams B31 to B51, shown in FIG. 4, in the second scan. By subjecting the two optical tomographic images, which have been obtained by measurement performed at the positions of two points of different height, to combining processing in the signal processing circuit 5, the optical tomographic images of the gum GU and tooth TO shown in FIG. 4 are obtained. Needless to say, the optical tomographic images Igu1, Igu2 and Ito of the upper half of gum GU and tooth TO and the optical tomographic images Igu1, Igu2 and Ito of the lower half of gum GU and tooth TO are combined so as to be superimposed with regard to the overlapping portions thereof, and the connectivity of the optical tomographic images in the vertical direction is assured so as to obtain optical tomographic images that will be identical to the optical tomographic images Igu1, Igu2 and Ito that would be obtained by a single scan.

In the foregoing embodiment, the second optical fibers 21 to 25 are arrayed in a single row but they may be arrayed in two rows or more. In such case it may be arranged so as to be able to deflect the measuring light LM in the directions of two dimensions and arranged so as to guide the measuring light to the optical fibers included in each row. Further, the five optical fibers 21 to 25 need not necessarily be arrayed on a straight line and may be arranged along a curving line.

FIGS. 7 to 12 illustrate an example of the examination head 20.

FIGS. 7 to 12 are depicted in a form flipped vertically in comparison with FIGS. 1 and 4. If we assume that FIGS. 7 to 12 represent the upright state (or the inverted state), then FIGS. 1 and 4 represent the inverted state (or the upright state).

With reference to FIG. 7, a slide groove 52 is formed in a slide surface 51 of the holder 50 having the shape of a rectangular parallelepiped. The light-emitting member 30 and the reflecting member 40 are held by the holder 50 on the slide surface 51 so as to be freely slidable. The light-emitting member 30 has a three-layer structure in which a deforming member 32 consisting of a resin freely deformable by application of pressure is sandwiched between the comparatively rigid plate-shaped member 31 and a freely deformable plate-shaped member 33. The plate-shaped member 33 deforms tracking the deformation of the deforming member 32. In FIG. 7, the front surface of the one plate-shaped member 31 serves as the light-incident surface 31A, and the back surface of the other plate-shaped member 33 serves as the light-emitting surface 33A that emits the measuring light beams B11 to B51. Further, the reflecting member 40 also has a three-layer structure in which the deforming member 42 consisting of a resin freely deformable by application of pressure is sandwiched between the sheet-like mirror 41 and the comparatively rigid plate-shaped member 43. In FIG. 7, the front surface of the sheet-like mirror 41 serves as the reflecting surface. The light-emitting member 30 includes the deforming member 32, and the reflecting member 40 includes the deforming member 42. In a case where the tooth TO and gum GU are interposed between the light-emitting member 30 and the reflecting member 40, therefore, the tooth TO or gum GU comes into close contact with the light-emitting surface 33A and reflecting surface 41A. The light-emitting member 30 and the reflecting member 40 need not necessarily be three-layer structures and need not be deformable as by the deforming members 32, 42. Further, the end face of the deforming member 32 may be adopted as the light-emitting surface and not the plate-shaped member 33 of the light-emitting member 30 and may emit the measuring light beams B11 to B51. Furthermore, the light-emitting surface 33A and reflecting surface 41A need not come into close contact with the tooth TO or gum GU, and the light-emitting member 30 and reflecting member 40 need not necessarily be deformed.

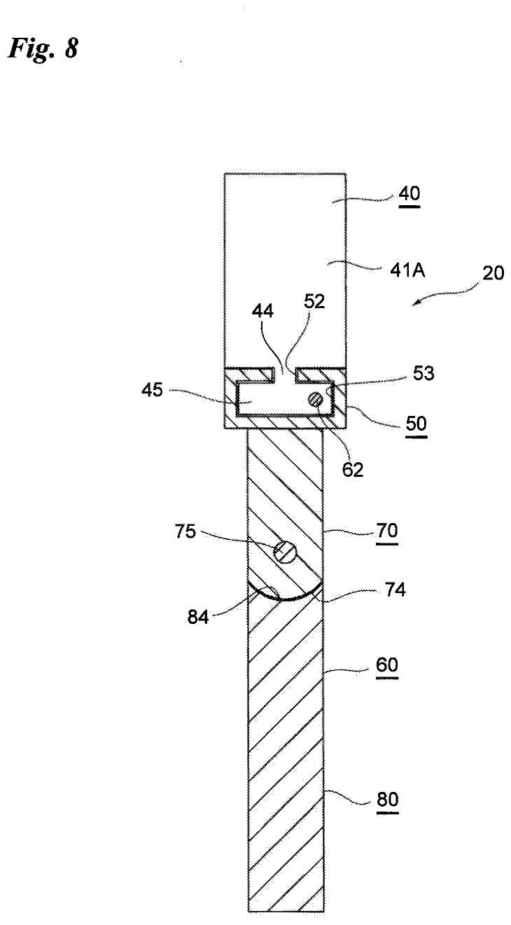

FIG. 8 is a sectional view taken along line VIII-VIII of FIG. 7.

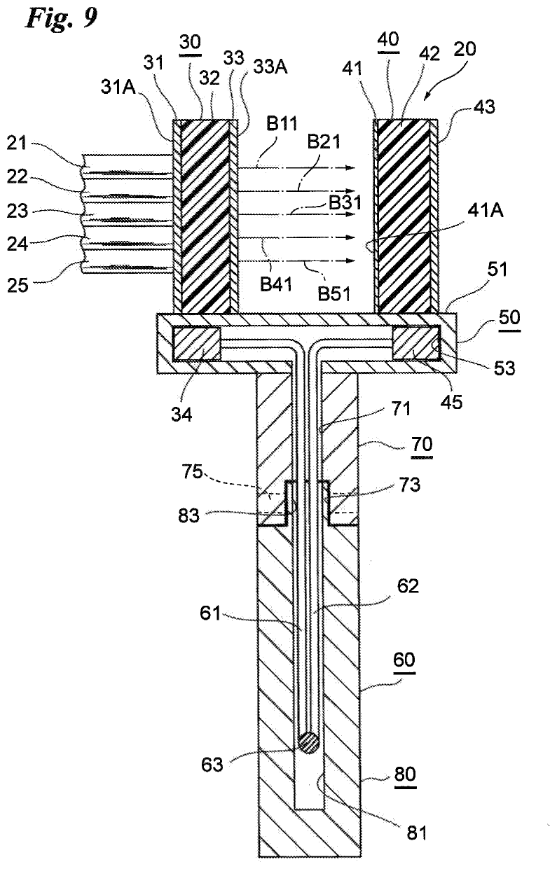

The inside of the holder 50 is a cavity 53 the upper part of which constitutes the slide groove 52. A portion of the lower part of the reflecting member 40 is a neck 44 of small width, and a portion lower than the neck 44 is a sliding portion 45 having a width larger than that of the neck 44. The neck 44 is received inside the slide groove 52 and the reflecting member 40 slides along the slide groove 52. Since the width of the sliding portion 45 is larger than the width of the slide groove 52, the reflecting member 40 slides along the slide groove 52 without coming off the holder 50. Of two transfer members 61, 62 (see FIG. 9) described later, the latter transfer member 62 is secured to the sliding portion 45.

The light-emitting member 30 also has a construction similar to that of the reflecting member 40, and the light-emitting member 30 also slides along the slide groove 52.

Returning to FIG. 7, the second optical fibers 21 to 25 are inserted into the light-incident surface 31A on the outer side of the light-emitting member 30, and the measuring light beams B11 to B51 are emitted from the light-emitting surface 33A on the side opposite the light-incident surface 31A. The measuring light beams B11 to B51 are reflected at the reflecting surface 41A of the reflecting member 40, as described above. The light-emitting surface 33A of the light-emitting member 30 and the reflecting surface 41A of the reflecting member 40 are held on the holder 50 so as to lie parallel.

The gripping member 60 which is rod-shaped and secured to the lower face of the holder 50 extends downwardly from the holder 50. The holder 50 includes a fixed portion 70 secured to the holder 50, and a folding portion 80 that folds through a predetermined angle about a folding shaft 75 (one example of an axis of rotation) secured to the holder 50. A recess 73 is formed in the lower portion of the fixed portion 70, and a protruding portion 83 formed on the upper portion of the folding portion 80 is received inside the recess 73. The lower face of the fixed portion 70 defines an arcuate face 74 formed in the shape of a convex arc. The upper face of the folding portion 80 defines an arcuate face 84 formed in the shape of a concave arc. The arcuate face 74 of the fixed portion 70 and the arcuate face 84 of the folding portion 80 face each other, and the recess 73 receives the protruding portion 83 formed on the upper portion of the folding portion 80. The folding shaft 75 penetrates the recess 73 and protruding portion 83 in such a manner that the direction of the central axis of the folding shaft 75 takes on a direction that is the same as the direction of emission of the measuring light beams B11 to B51. As a result, the folding portion 80 is free to fold, with respect to the fixed portion 70, about the folding shaft 75.

An operating button 64 is formed in the surface of the folding portion 80. The light-emitting member 30 and reflecting member 40 are made to separate from each other by manipulating the operating button 64 upward, and the light-emitting member 30 and reflecting member 40 are made to approach each other by manipulating the operating button 64 downward (though the actions may just as well be the converse of those described).

FIG. 9 is a sectional view taken along line IX-IX of FIG. 7.

With reference to FIG. 9, the fixed portion 70 is formed to have a hollow portion 71 connected to the cavity 53 within the holder 50 and extending in the downward direction in FIG. 7. The folding portion 80 also is formed to have a hollow portion 81 extending in the downward direction. The two transfer members 61 and 62 pass through the hollow portions 81, 71 and into the interior of the cavity 53. The tip of one transfer member 61 is secured to a sliding portion 34 of the light-emitting member 30, and the aft end of the transfer member is secured to an operating shaft 63 connected to the operating button 64. The tip of the other transfer member 62 is secured to the sliding portion 45 of the reflecting member 40, and the aft end of this transfer member is secured to the operating shaft 63 connected to the operating button 64. These two transfer members 61 and 62 are constituted by resin or springs or the like which, though flexible, are capable of transferring a force that pushes the light-emitting member 30 and reflecting member 40 if a pushing force is applied.

When the operating button 64 is raised, the force that pushes up the operating button 64 acts on the operating shaft 63 of the operating button 64. The pushing force is applied to the light-emitting member 30 by one transfer member 61 secured to the operating shaft 63, and the pushing force is applied to the reflecting member 40 by the other transfer member 62 secured to the shaft 63. The forces act in directions that result in the light-emitting member 30 and reflecting member 40 separating from each other. As shown in FIGS. 7 and 9, the light-emitting member 30 and reflecting member 40 separate while the light-emitting surface 33A and reflecting surface 41A maintain the parallel state. In this case, the transfer members 61 and 62 function as second transfer members.

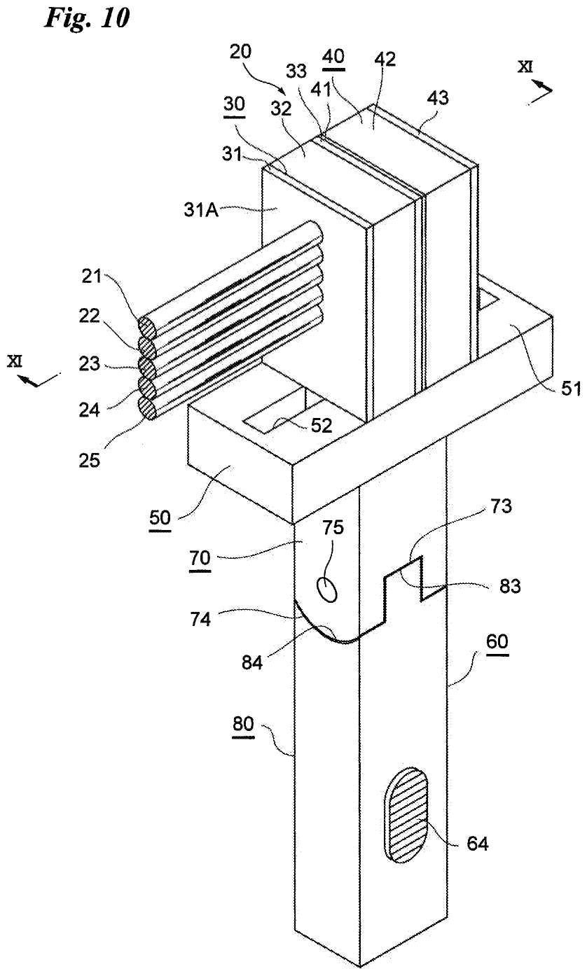

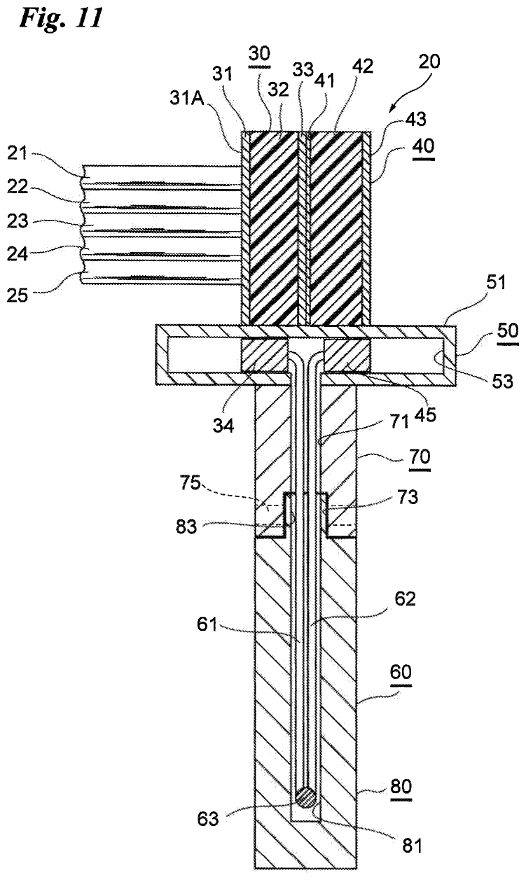

FIG. 10 is a perspective view of the examination head 20 and illustrates a state in which the light-emitting member 30 and reflecting member 40 are close together. FIG. 11 is a sectional view taken along line XI-XI of FIG. 10.

When the operating button 64 is lowered, the two transfer members 61, 62 are pulled downward. The light-emitting member 30 is pulled by one transfer member 61, and the reflecting member 40 is pulled by the other transfer member 62. As a result, the light-emitting member 30 and reflecting member 40 approach each other while the light-emitting surface 33A and reflecting surface 41A maintain the parallel state. In this case, the transfer members 61 and 62 function as first transfer members.

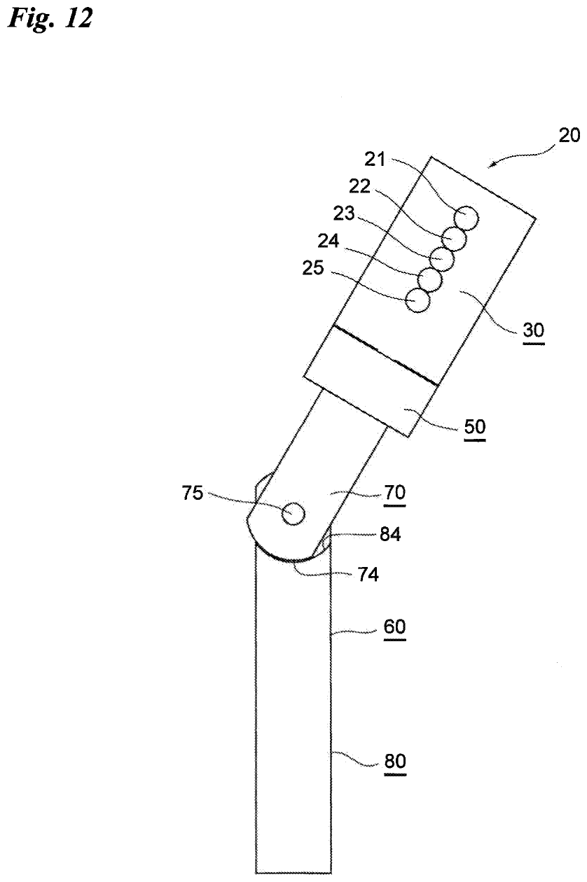

FIG. 12 is a side view illustrating the gripping member 60 in the folded state.

In a case where the tooth TO and gum GU are sandwiched between the light-emitting member 30 and the reflecting member 40 of the examination head 20, there are instances where the gripping member 60 becomes a hindrance, making it difficult to sandwich the tooth and gum. For this reason the gripping member 60 is made freely foldable about the folding shaft 75.

In the example shown in FIGS. 1 to 12, the direction of the axis of the folding shaft 75 is assumed to be identical with the direction of emission of the measuring light beams B11 to B51. However, the gripping member 60 may be secured to the holder 50 in such a manner that the axis of the folding shaft 75 will form a right angle with the optic axis of measuring light beams B11 to B51 centered about the axis of the gripping member 60 in the longitudinal direction thereof. In FIG. 7 and the like, the gripping member 60 is capable of being folded in such a manner that the light-emitting member 30 it tilted forward or backward.

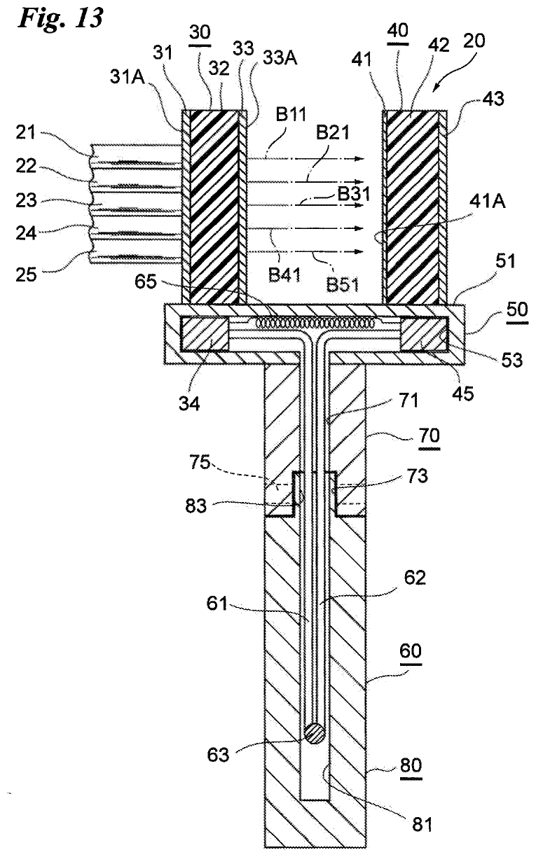

FIG. 13, which illustrates a modification, is a sectional view of the examination head 20 and corresponds to FIG. 9. Components in FIG. 13 identical with those shown in FIG. 9 and the like are designated by like symbols and a description thereof is omitted.

In the examination head 20 shown in FIG. 13, a spring 65 is provided in the cavity 53 of the holder 50 and has its two ends connected to respective ones of the sliding portion 34 of light-emitting member 30 and the sliding portion 45 of the reflecting member 40.

The spring 65 may be a compression spring or a tension spring. If, in a case where the spring 65 is a compression spring (one example of a compression member), a force is not acting on the operating button 64, the light-emitting member 30 and reflecting member 40 will be separate from each other and the operating button 64 will be in the raised state. If the operating button 64 is lowered against the force of the spring 65, the light-emitting member 30 and reflecting member 40 are pulled by the force of the two transfer members 61 and 62 (which function as first transfer members) and approach each other. If the operating button 64 is released, the light-emitting member 30 and reflecting member 40 separate owing to the compressive force of the spring 65. In the case where the spring is a compression spring, it will suffice if a tensile force is applied in such a manner that the light-emitting member 30 and reflecting member 40 approach each other. The transfer members 61 and 62 may therefore be cord-like. If, in a case where the spring 65 is a tension spring (one example of a tension member), a force is not acting on the operating button 64, the light-emitting member 30 and reflecting member 40 will be close together and the operating button 64 will be in the lowered state. If the operating button 64 is raised against the force of the spring 65, the light-emitting member 30 and reflecting member 40 are pushed by the force of the two transfer members 61 and 62 (which function as second transfer members) and separate from each other. If the operating button 64 is released, the light-emitting member 30 and reflecting member 40 approach it other owing to the tensile force of the spring 65. It should be noted that thus far an embodiment has been described in which the light-emitting member 30 and reflecting member 40 are capable of approaching each other and separating owing to the fact that examination head 20A is equipped with the light-emitting member 30 and reflecting member 40 in such a manner that both are slidable. However, it may be arranged so that the light-emitting member 30 and reflecting member 40 are made capable of approaching and separating by fixing one of either the light-emitting member 30 or reflecting member 40 to holder 50A and equipping the examination head 20A with the other of the light-emitting member 30 or reflecting member 40 in slidable fashion.

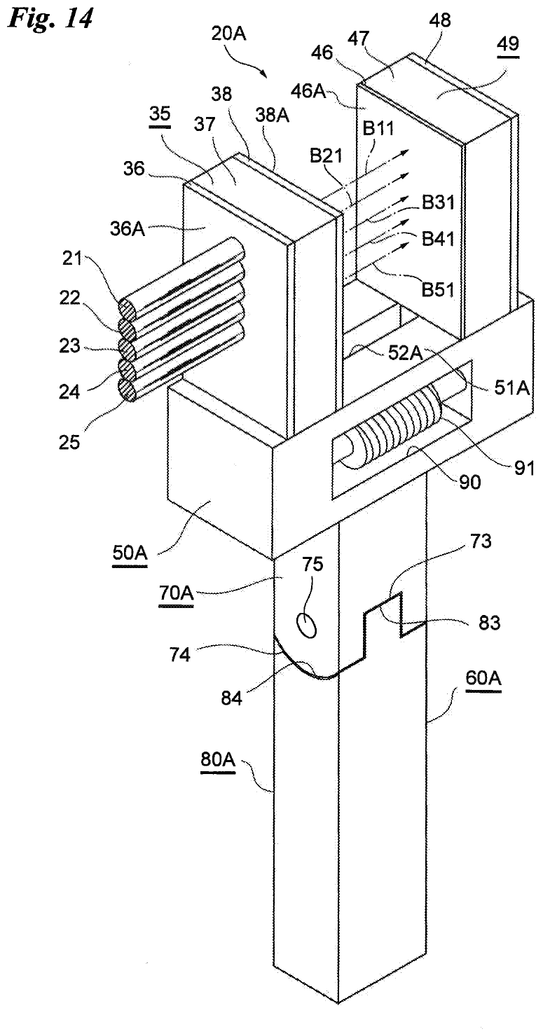

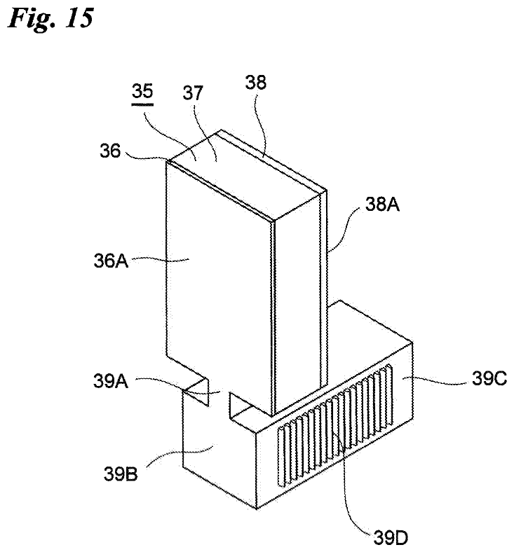

FIGS. 14 and 15 illustrate another modification.

FIG. 14 is a perspective view of examination head 20A.

Holder 50A of the examination head 20A is formed to have a slide groove 52A in a slide surface 51A along the longitudinal direction of the holder 50A. A light-emitting member 35 is such that a deforming member 37 is sandwiched between a plate-shaped member 36 and a freely deformable plate-shaped member 38. The plate-shaped member 38 is deformed tracking the deformation of the deforming member 37. The second optical fibers 21 to 25 are inserted into a light-incident surface 36A of the light-emitting member 35, and the measuring light beams B11 to B51 are emitted from the light-emitting surface 38A. The slide groove 52A is formed in such a manner that the direction of emission of the measuring light beams B11 to B51 and the sliding direction of the light-emitting member 35 are the same. A reflecting member 49 is secured to one end of the holder 50A. The reflecting member 49 is such that a deforming member 47 is sandwiched between a sheet-like mirror 46 and a plate-shaped member 48. The surface of the sheet-like mirror 46 serves as a reflecting surface 46A. In the examination head 20A shown in FIG. 14, the reflecting member 49 does not slide but may just as well be slidable. Further, the light-emitting member 35 may be secured to one end of the holder 50A and the reflecting member 49 may be slidable.

The interior of the holder 50A is formed to have a cavity of the kind shown in FIGS. 9 and the like in order to allow the light-emitting member 35 to slide. The side face of the holder 50A is formed to have an opening 90. A worm 91 is mounted freely rotatably inside the opening 90.

FIG. 15 is a perspective view of the light-emitting member 35.

The light-emitting member 35 is formed to have a neck 39A of small width in a manner similar to that of the reflecting member 40 shown in FIG. 8, etc., and the neck 39A is formed to have a sliding portion 39B. The sliding portion 39B extends along the direction of emission of the measuring light beams B21 to B25, and the light-emitting member 35 has a substantially L-shaped configuration as viewed from the side face. A rack 39D is formed in the sliding portion 39B in the lower portion of side face 39C.

When the light-emitting member 35 is mounted on the holder 50A, teeth on the worm 91 of the holder 50A and teeth on the rack 39D of the light-emitting member 35 mesh. By rotating the worm 91, the light-emitting member 35 slides along the longitudinal direction of the holder 5A and the light-emitting member 35 and reflecting member 49 approach each other and separate from each other.

A gripping member 60A is secured to the lower surface of the holder 50A. The gripping member 60A includes fixed portion 70A secured to the holder 50A, and a folding portion 80A which, owing to the folding shaft 75, folds freely with respect to the fixed portion 70A.

By virtue also of the examination head 20A of the kind shown in FIGS. 14 and 15, the light-emitting surface 38A of the light-emitting member 35 and the reflecting surface 46A of the reflecting member 49 are capable of approaching and separating while they maintain a parallel state. In the embodiment described above, the tooth TO or the tooth TO and gum GU are irradiated with the measuring light beams B21 to B25 using the five optical fibers 21 to 25. However, it may be arranged so that, rather than provide the five optical fibers 21 to 25, the tooth TO or the tooth TO and gum GU are irradiated directly with the measuring light LM emitted from the first optical fiber 7. In a case where the tooth TO or the tooth TO and gum GU are irradiated with the measuring light LM emitted from the first optical fiber 7 in such an arrangement, the measuring-light-LM light-emitting end face of the first optical fiber 7 is connected to the examination head 20 and the examination head 20 is moved up and down (along the Z and −Z directions in FIG. 1), whereby multiple measuring light beams LM, which correspond to the above-mentioned measuring light beams B11 to B51, and reflected light beams, which correspond to the reflected light beams R11 to R51, are obtained. That is, it will suffice if the tooth TO and gum GU are irradiated with light such as the measuring light LM and measuring light beams B21 to B25; the optical fibers 21 to 25 need not be utilized. Further, in the deflecting unit 10 shown in FIG. 2, the measuring light LM is distributed to the five optical fibers 21 to 25 using the deflecting mirror 13. However, the measuring light LM may be distributed to the five optical fibers 21 to 25 using a liquid crystal deflecting element (not shown). The incident measuring light LM is deflected in accordance with a voltage impressed upon the liquid crystal deflecting element. Furthermore, although the optical fibers 21 to 25 are connected to the deflecting unit 10 in the above-described embodiment, the optical fibers 21 to 25 may be incorporated within the deflecting unit 10 and the deflecting unit 10 and light-emitting member 30 may be integrated.

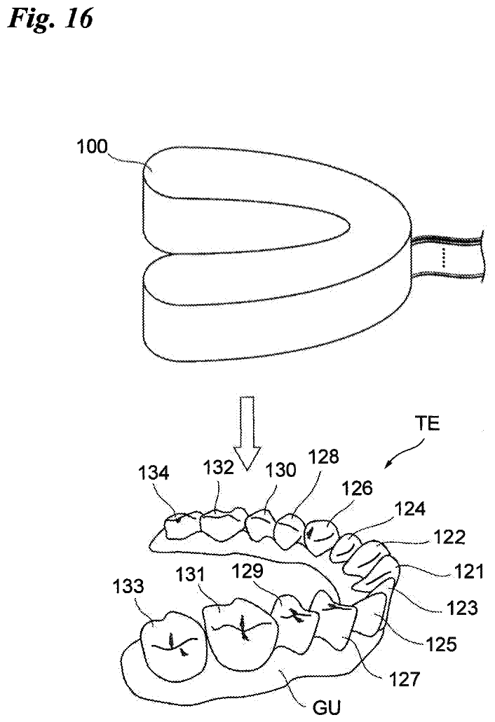

FIGS. 16 to 22, which illustrate another embodiment, show an example of a mouthpiece 100 serving as an examination head. Specifically, the mouthpiece 100 corresponds to one mode of the holder.

The upper part of FIG. 16 is a perspective view of the mouthpiece 100, and the lower part a perspective view of gums GU and lower teeth TE (central incisors 121 and 122, lateral incisors 123 and 124, canines 125 and 126, first premolars 127 and 128, second premolars 129 and 130, first molars 131 and 132, and second molars 133 and 134) on which the mouthpiece 100 is placed.

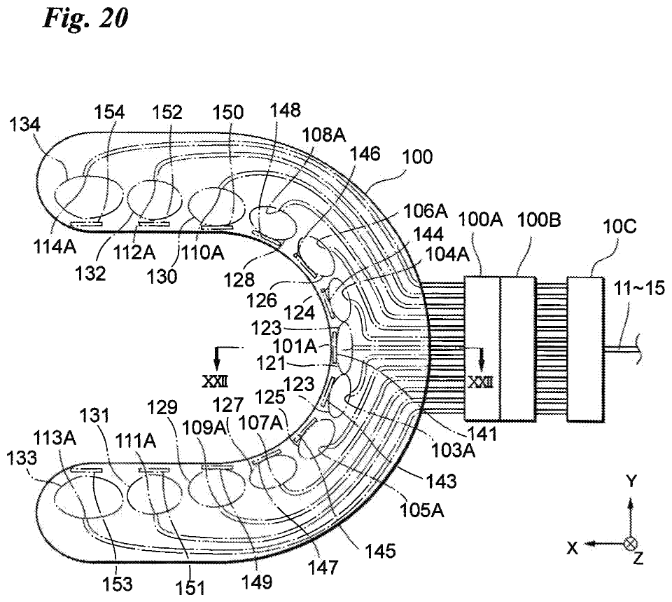

As will be described below in detail (refer to FIGS. 18, 20 and the like), the interior of the mouthpiece 100 includes a plurality of optical fibers. The mouthpiece 100 is made of a flexible material and holds the plurality of optical fibers, which are included in the mouthpiece 100, freely movable independently along the direction of the optical axes of the optical fibers. They need not be freely movable, though.

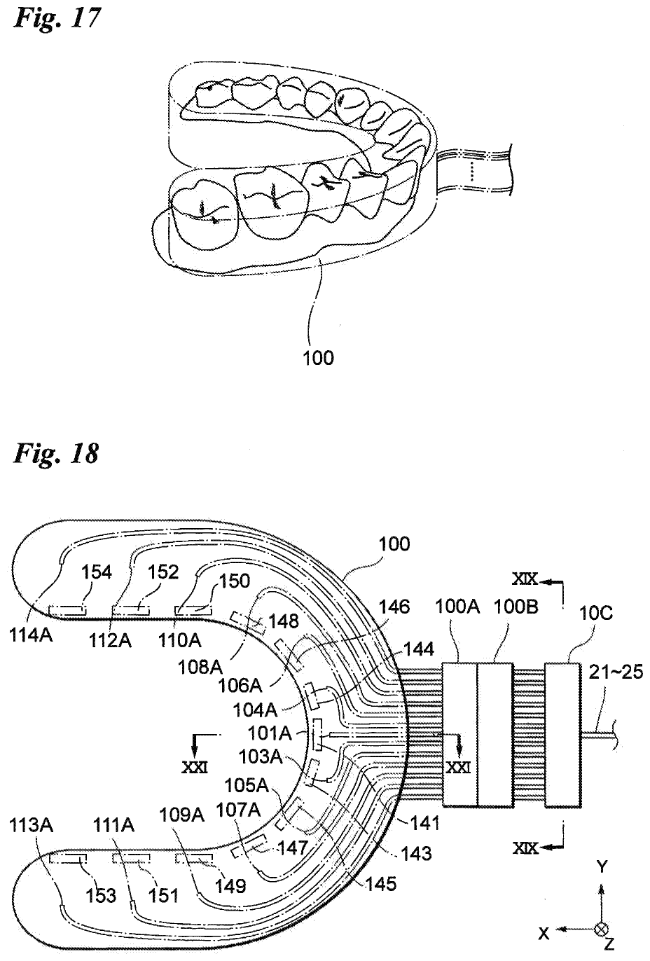

FIG. 17 illustrates the manner in which the mouthpiece 100 is placed on teeth TE and gums GU.

A cavity is formed inside the mouthpiece 100, and the inner surface of the mouthpiece 100 is placed in close contact with the surface of the teeth TE and the surface of the gums GU.

FIG. 18 is a plan view of the mouthpiece 100.

The mouthpiece 100 includes a multiplicity of optical fibers such as optical fibers 101A to 114A. (The optical fibers 101A to 114A and the like are indicated by broken lines in order to facilitate understanding.) The multiplicity of optical fibers extend to the exterior of the mouthpiece 100 from the front (the right side in FIG. 17) thereof. The multiplicity of optical fibers are separably coupled by a pair of connectors (connector 100A and connector 100B).

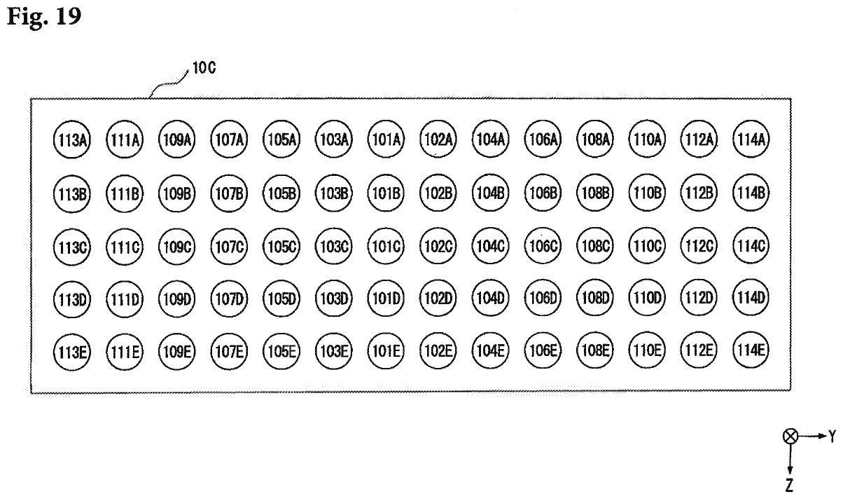

The optical fibers such as the optical fibers 101A to 114A that extend from the connector 100B are connected to one end of the deflecting unit 10C, and the five optical fibers 21 to 25 are connected to the other end of the deflecting unit 10C. Using the deflecting mirror provided within the deflecting unit 10C, the deflecting unit 10C deflects the measuring light LM, which is output from optical fibers 21 to 25, causing the measuring light LM to propagate toward optical fibers 101A to 101E, 102A to 102E, 103A to 103E, 104A to 104E, 105A to 105E, 106A to 106E, 107A to 107E, 108A to 108E, 109A to 109E, 110A to 110E, 111A to 111E, 112A to 112E, 113A to 113E, or 114A to 114E (see FIG. 19).

Reflecting members 141 to 154 and the like are disposed via the cavity at positions confronting the other end faces of the optical fibers 101A to 114A (these end faces are the faces of the ends not connected to the connector 100A; they are light-emitting end faces which emit measuring light beams that are parallel light beams).

FIG. 19 is a sectional view taken along line XIX-XIX of FIG. 16. Hatching is omitted in FIG. 19.

The multiplicity of optical fibers 101A to 101E, 102A to 102E, 103A to 103E, 104A to 104E, 105A to 105E, 106A to 106E, 107A to 107E, 108A to 108E, 109A to 109E, 110A to 110E, 111A to 111E, 112A to 112E, 113A to 113E and 114A to 114E, which extend to the outside from the mouthpiece 100, are connected to the deflecting unit 10C. The multiplicity of optical fibers 101A to 101E, 102A to 102E, 103A to 103E, 104A to 104E, 105A to 105E, 106A to 106E, 107A to 107E, 108A to 108E, 109A to 109E, 110A to 110E, 111A to 111E, 112A to 112E, 113A to 113E and 114A to 114E are arrayed in respective rows along the direction of the Z-axis (up-and-down direction).

The measuring light LM emitted from among the optical fibers 21 to 25 is deflected toward the optical fibers 101A to 101E, 102A to 102E, 103A to 103E, 104A to 104E, 105A to 105E, 106A to 106E, 107A to 107E, 108A to 108E, 109A to 109E, 110A to 110E, 111A to 111E, 112A to 112E, 113A to 113E, or 114A to 114E by the deflecting unit 10C. The deflecting unit 10C shown in FIGS. 18 and 20 thus deflects the measuring light LM, which has been emitted from among the optical fibers 21 to 25, in the direction of one dimension. However, the deflecting unit 10C may deflect the measuring light LM, which has been emitted from the first optical fiber 7, in the directions of two dimensions. In FIG. 18 or 20, the first optical fiber 7, instead of the five optical fibers 21 to 25, may be connected to the deflecting unit 10C. In this case, since a deflecting unit for deflection from the first optical fiber 7 to the five optical fibers 21 to 25 will no longer be necessary, the deflecting units provided for the overall periodontal disease examination apparatus will now be a single unit.

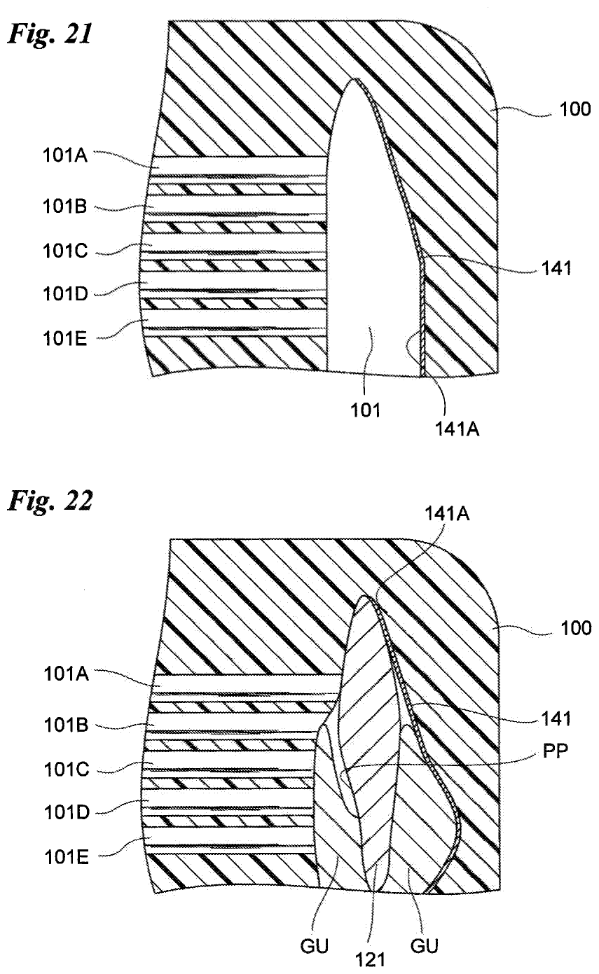

FIG. 20 is a plan view illustrating the manner in which the mouthpiece 100 is placed on the teeth TE and gums GU. FIG. 21 is a sectional view taken along line XXI-XXI of FIG. 18, and FIG. 22 is a sectional view taken along line XXII-XXII of FIG. 20.

The optical fibers 101A to 101E arrayed in a single row are held by the mouthpiece 100 such that the light-emitting surfaces of the optical fibers 101A to 101E are exposed at a close-contact surface of the teeth TE and gums GU.

Reflecting member 141 is disposed at a position confronting the light-emitting end faces of the optical fibers 101A to 101E. The surface of the reflecting member 141 serves as a reflecting surface 141A. The mouthpiece 100 consists of an elastic deformable resin or the like. When the teeth TE and gums GU are inserted into a space 101 between the light-emitting end faces of the optical fibers 101A to 101E and the reflecting surface 141A, the surfaces of the teeth TE or the gums GU come into close contact with the light-emitting end faces of the optical fibers 101A to 101E, and the back surfaces of the teeth TE or the gums GU come into close contact with the reflecting surface 141A.

When the mouthpiece 100 is placed on the teeth TE and gums GU, the light-emitting surfaces (on the right side in FIG. 22) of the optical fibers 101A to 101E come into close contact with the surface of gum GU, which envelops the central incisor 121, and with the surface of the central incisor 121 on its outer side, as illustrated in FIGS. 20 and 22.

Similarly, when the mouthpiece 100 is placed on the teeth TE and gums GU, the light-emitting surfaces of the optical fibers 102A to 102E come into close contact with the gum GU, which envelops the central incisor 122, and with the surface of the central incisor 122 on its outer side, the light-emitting surfaces of the optical fibers 103A to 103E come into close contact with the gum GU, which envelops the lateral incisor 123, and with the surface of the lateral incisor 123 on its outer side, the light-emitting surfaces of the optical fibers 104A to 104E come into close contact with the gum GU, which envelops the lateral incisor 124, and with the surface of the lateral incisor 124 on its outer side, the light-emitting surfaces of the optical fibers 105A to 105E come into close contact with the gum GU, which envelops the canine 125, and with the surface of the canine 125 on its outer side, and the light-emitting surfaces of the optical fibers 106A to 106E come into close contact with the gum GU, which envelops the canine 126, and with the surface of the canine 126 on its outer side. Further, the light-emitting surfaces of the optical fibers 107A to 107E come into close contact with the gum GU, which envelops the first premolar 127, and with the surface of the first premolar 127 on its outer side, the light-emitting surfaces of the optical fibers 108A to 108E come into close contact with the gum GU, which envelops the first premolar 128, and with the surface of the first premolar 128 on its outer side, the light-emitting surfaces of the optical fibers 109A to 109E come into close contact with the gum GU, which envelops the second premolar 129, and with the surface of the second premolar 129 on its outer side, the light-emitting surfaces of the optical fibers 110A to 110E come into close contact with the gum GU, which envelops the second premolar 130, and with the surface of the second premolar 130 on its outer side, the light-emitting surfaces of the optical fibers 111A to 111E come into close contact with the gum GU, which envelops the first molar 131, and with the surface of the first molar 131 on its outer side, the light-emitting surfaces of the optical fibers 112A to 112E come into close contact with the gum GU, which envelops the first molar 132, and with the surface of the first molar 132 on its outer side, the light-emitting surfaces of the optical fibers 113A to 113E come into close contact with the gum GU, which envelops the second molar 133, and with the surface of the second molar 133 on its outer side, and the light-emitting surfaces of the optical fibers 114A to 114E come into close contact with surface of the second molar 134 on its outer side.

When the measuring light LM emitted from the second optical fibers 21 to 25 is deflected and impinges on the optical fibers 101A to 101E, the measuring light LM irradiates the gum GU, which envelops the central incisor 121, as well as the central incisor 121, and is reflected from the central incisor 121 and gum GU. Further, the measuring light LM that has passed through the gum GU or central incisor 121 irradiates the reflecting surface 141A and is reflected from the reflecting surface 141A. The measuring light LM reflected from the central incisor 121, the gum GU and the reflecting surface 141A, etc., returns to the optical fibers 101A to 101E that emitted the measuring light, propagates through the optical fibers 101A to 101E and, as described above, impinges upon the photodiode 4 along with the reference light LR, whereby optical tomographic images of the gum GU enveloping the central incisor 121 and of the central incisor 121 are obtained. Since the measuring light LM reflected from the reflecting surface 141A is utilized, interference signals having an excellent S/N ratio are obtained and so are optical tomographic images that are comparatively easy to observe.

When the measuring light LM emitted from the second optical fibers 21 to 25 is deflected and impinges on the optical fibers 102A to 102E, the measuring light LM irradiates the gum GU, which envelops the central incisor 122, as well as the central incisor 122, and optical tomographic images of the gum GU enveloping the central incisor 122 and of the central incisor 122 are therefore obtained. When the measuring light LM emitted from the second optical fibers 21 to 25 is deflected and impinges on the optical fibers 103A to 103E, the measuring light LM irradiates the gum GU, which envelops the lateral incisor 123, as well as the lateral incisor 123, and optical tomographic images of the gum GU enveloping the lateral incisor 123 and of the lateral incisor 123 are therefore obtained. When the measuring light LM emitted from the second optical fibers 21 to 25 is deflected and impinges on the optical fibers 104A to 104E, the measuring light LM irradiates the gum GU, which envelops the lateral incisor 124, as well as the lateral incisor 124, and optical tomographic images of the gum GU enveloping the lateral incisor 124 and of the lateral incisor 124 are therefore obtained. When the measuring light LM emitted from the second optical fibers 21 to 25 is deflected and impinges on the optical fibers 105A to 105E, the measuring light LM irradiates the gum GU, which envelops the canine 125, as well as the canine 125, and optical tomographic images of the gum GU enveloping the canine 125 and of the canine 125 are therefore obtained. When the measuring light LM emitted from the second optical fibers 21 to 25 is deflected and impinges on the optical fibers 106A to 106E, the measuring light LM irradiates the gum GU, which envelops the canine 126, as well as the canine 126, and optical tomographic images of the gum GU enveloping the canine 126 and of the canine 126 are therefore obtained.

Similarly, when the measuring light LM emitted from the second optical fibers 21 to 25 is deflected and impinges on the optical fibers 107A to 107E, the measuring light LM irradiates the gum GU, which envelops the first premolar 127, as well as the first premolar 127, and optical tomographic images of the gum GU enveloping the first premolar 127 and of the first premolar 127 are therefore obtained. When the measuring light LM emitted from the second optical fibers 21 to 25 is deflected and impinges on the optical fibers 108A to 108E, the measuring light LM irradiates the gum GU, which envelops the first premolar 128, as well as the first premolar 128, and optical tomographic images of the gum GU enveloping the first premolar 128 and of the first premolar 128 are therefore obtained. When the measuring light LM emitted from the second optical fibers 21 to 25 is deflected and impinges on the optical fibers 109A to 109E, the measuring light LM irradiates the gum GU, which envelops the second premolar 129, as well as the second premolar 129, and optical tomographic images of the gum GU enveloping the second premolar 129 and of the second premolar 129 are therefore obtained. When the measuring light LM emitted from the second optical fibers 21 to 25 is deflected and impinges on the optical fibers 110A to 110E, the measuring light LM irradiates the gum GU, which envelops the second premolar 130, as well as the second premolar 130, and optical tomographic images of the gum GU enveloping the second premolar 130 and of the second premolar 130 are therefore obtained.

Furthermore, when the measuring light LM emitted from the second optical fibers 21 to 25 is deflected and impinges on the optical fibers 111A to 111E, the measuring light LM irradiates the gum GU, which envelops the first molar 131, as well as the first molar 131, and optical tomographic images of the gum GU enveloping the first molar 131 and of the first molar 131 are therefore obtained. When the measuring light LM emitted from the second optical fibers 21 to 25 is deflected and impinges on the optical fibers 122A to 122E, the measuring light LM irradiates the gum GU, which envelops the first molar 132, as well as the first molar 132, and optical tomographic images of the gum GU enveloping the first molar 132 and of the first molar 132 are therefore obtained. When the measuring light LM emitted from the second optical fibers 21 to 25 is deflected and impinges on the optical fibers 133A to 133E, the measuring light LM irradiates the gum GU, which envelops the second molar 133, as well as the second molar 133, and optical tomographic images of the gum GU enveloping the second molar 133 and of the second molar 133 are therefore obtained. When the measuring light LM emitted from the second optical fibers 21 to 25 is deflected and impinges on the optical fibers 114A to 114E, the measuring light LM irradiates the gum GU, which envelops the second molar 134, as well as the second molar 134, and optical tomographic images of the gum GU enveloping the second molar 134 and of the second molar 134 are therefore obtained.

With regard to the optical fibers in addition to the optical fibers 101A to 101E, reflecting members 142, 143, 144, 145, 146, 147, 148, 149, 150, 151, 152, 153 and 154 also are provided confronting the tip-side light-emitting end faces of the optical fibers 102A to 102E, 103A to 103E, 104A to 104E, 105A to 105E, 106A to 106E, 107A to 107E, 108A to 108E, 109A to 109E, 110A to 110E, 111A to 111E, 112A to 112E, 113A to 113E and 114A to 114E. Since interference signals are generated utilizing the light reflected from these reflecting members 142 to 154, interference signals having an excellent S/N ratio can be generated and optical tomographic images that are comparatively easy to observe are obtained.

By placing the mouthpiece 100 on the teeth TE and gums GU and causing the measuring light LM to propagate toward the second optical fibers 21 to 25, the measurer can detect the depths of multiple periodontal pockets corresponding to multiple teeth TE without manually performing alignment successively with respect to each individual tooth TO undergoing measurement and the gum that contains each tooth TO. As a result, in comparison with a case where the measurer performs such alignment successively with respect to each periodontal pocket corresponding to each individual tooth TO, it is possible to achieve a reduction in measurer inconvenience and a shorter measurement time.

The above-described mouthpiece 100 can be produced by introducing the multiplicity of optical fibers into a previously prepared mold for the mouthpiece 100 and pouring in a resin of flexible material. Alternatively, the above-described mouthpiece 100 may be produced by molding the shape of the mouthpiece 100 using a resin of flexible material, thereafter forming space portions, and passing the multiplicity of optical fibers through these space portions. Further, regardless of which method of production is used, preferably a GRIN lens is provided on the tip of each of the multiplicity of optical fibers 101A to 101E, 102A to 102E, 103A to 103E, 104A to 104E, 105A to 105E, 106A to 106E, 107A to 107E, 108A to 108E, 109A to 109E, 110A to 110E, 111A to 111E, 112A to 112E, 113A to 113E and 114A to 114E, or the tip of each of the multiplicity of optical fibers 101A to 101E, 102A to 102E, 103A to 103E, 104A to 104E, 105A to 105E, 106A to 106E, 107A to 107E, 108A to 108E, 109A to 109E, 110A to 110E, 111A to 111E, 112A to 112E, 113A to 113E and 114A to 114E is machined to thereby make it a parallelizing element.

In the foregoing embodiment, the mouthpiece 100 for the lower jaw is described. However, the depths of periodontal pockets can be detected in similar fashion with a mouthpiece 100 for the upper jaw rather than the lower jaw. Further, it may be so arranged as to provide optical fibers inside the mouthpiece 100 so as to detect the depths of a periodontal pockets on the inner-side surface of the teeth TE rather than the depths of a periodontal pockets on the outer-side surface of the teeth TE. In such case the optical fibers would be provided inside the mouthpiece 100 such that their light-emitting surfaces will come into contact with inner-side surface of the teeth TE. Furthermore, in the foregoing embodiment, it is so arranged that the light-emitting end faces of the optical fibers of one row come into contact with one tooth. However, it may be so arranged that the light-emitting end faces of the optical fibers of two or more rows come into contact with one tooth.

In the foregoing embodiment, the detachable connectors 100A and 100B are provided on the tip-end side relative to the deflecting unit 10C. However, connectors (one example of a coupling member) may be provided between the tips of the optical fibers 21 to 25, which are shown in figures such as FIG. 1, and the base ends of the optical fibers 21 to 25 for coupling each of the optical fibers 21 to 25 in freely separable fashion. In any case, the mouthpiece 100 (examination head) can be detached from the base ends of the optical fiber array and the mouthpiece 100 can be replaced comparatively simply. As a result, owing to the fact that it is possible to detach the part that includes the tip of the optical fiber array which comes into contact with the oral cavity of the subject, it is possible to discard this part and replace it with an unused part on a per-subject basis. The degree of hygiene in examination of periodontal disease, therefore, can be improved. Furthermore, by virtue of the fact that the discarded portion does not include comparatively expensive members such as the deflecting unit, running cost is reduced in comparison with a case where these members are included in the discarded portion.

1. An examination head used in an apparatus for examining periodontal disease by utilizing measuring light and reference light split off from low-interference light, said examination head having: a holder formed to include a light-emitting surface for emitting measuring light which is parallel light, and a reflecting surface parallel to said light-emitting surface for reflecting the measuring light emitted from said light-emitting surface.

2. An examination head according to claim 1, wherein said light-emitting surface and said reflecting surface are formed on a light-emitting member and on a reflecting member, respectively;
said holder holding said light-emitting member and said reflecting member.

3. An examination head according to claim 2, wherein said holder holds said light-emitting member and said reflecting member such that said light-emitting surface and said reflecting surface are capable of approaching each other and of separating from each other while maintaining a parallel state.

4. An examination head according to claim 2, further having a rod-shaped gripping member one end portion of which is secured to said holder;
said gripping member being freely foldable through a predetermined angle, a straight line in a direction identical with that of the optic axis of the measuring light serving as an axis of rotation.

5. An examination head according to claim 3, further having a first transfer member for transferring a force applied to at least one of said light-emitting member and said reflecting member in such a manner that said light-emitting surface and said reflecting surface approach each other.

6. An examination head according to claim 3, further having a second transfer member for transferring a force applied to at least one of said light-emitting member and said reflecting member in such a manner that said light-emitting surface and said reflecting surface separate from each other.

7. An examination head according to claim 6, further having a tension member for pulling said light-emitting member and said reflecting member in such a manner that said light-emitting surface and said reflecting surface approach each other.

8. An examination head according to claim 6, further having a compression member for applying a force that separates said light-emitting member and said reflecting member in such a manner that said light-emitting surface and said reflecting surface separate from each other.

9. An examination head according to claim 1, wherein said holder is a mouthpiece placed in close contact with a surface portion of teeth at a boundary with gums and with a portion of the gums, and comprises a flexible material;
said mouthpiece is formed to have a cavity into which the teeth and a portion of the gums penetrate owing to placement of the mouthpiece;
said light-emitting surface is formed on one surface of two surfaces that oppose each other sandwiching between them the teeth in said cavity, and said reflecting surface is formed on the other surface of the two surfaces.

10. An examination head according to claim 1, wherein said reflecting surface is a front-surface mirror or a back-surface mirror.

11. A periodontal disease examination apparatus comprising:
an examination head set forth in claim 1;
an optical divider for splitting low-interference light into measuring light and reference light;
a parallelizing element for rendering as parallel light the measuring light split off by said optical divider;
an optical waveguide for guiding the measuring light, which has been rendered as parallel light by said parallelizing element, to said examination head and causing the measuring light to be emitted from the light-emitting surface;
a photodetector for outputting an interference signal obtained by detecting reflected light, which is light reflected from a gum or tooth owing to irradiation of the gum or tooth with the measuring light emitted from the light-emitting surface of said examination head, reflected light which is a result of the emitted light emitted from light-emitting surface of said examination head being reflected from the reflecting surface of said examination head, and reflected light which is a result of the reference light split off by said optical divider being reflected by a reference surface; and
a periodontal pocket data generating device for generating data regarding depth of a periodontal pocket based on the interference signal output from said photodetector.
